(19) United States
(12) Patent Application Publication
(10) Pub. No.: US 2011/0250652 A1
(43) Pub. Date: Oct. 13, 2011

(54) PROCESS FOR PRODUCTION OF AN ENZYMATIC PREPARATION FOR HYDROLYSIS OF CELLULOSE FROM LIGNOCELLULOSIC RESIDUES AND APPLICATION THEREOF IN THE PRODUCTION OF ETHANOL

(75) Inventors: Aline Machado de Castro, Rio de Janeiro (BR); Lidia Maria Melo Santanna, Rio de Janeiro (BR); Nei Pereira Junior, Rio de Janeiro (BR); Absai Da Conceicao Gomes, Rio de Janeiro (BR); Emerson Pires Menezes, Rio de Janeiro (BR); Claudia Julia Groposo Silveira, Rio de Janeiro (BR); Danuza Nogueira Moyses, Rio de Janeiro (BR); Luiz Fernando Martins Bandeira, Rio de Janeiro (BR); Roberto Nobuyuki Maeda, Rio de Janeiro (BR)

(73) Assignee: PETROLEO BRASILEIRO S.A.- PETROBRAS, Rio de Janeiro (BR)

(21) Appl. No.: 13/002,171

(22) PCT Filed: Dec. 22, 2009
(86) PCT No.: PCT/GB09/02929

§ 371 Date: Jun. 8, 2011

(30) Foreign Application Priority Data
Dec. 29, 2008 (BR) PI0805560-2

(51) Int. Cl. C12P 19/14; C12N 9/96; C12P 7/02; C12N 9/42

(52) U.S. Cl. 435/99; 435/209; 435/188; 435/155

(57) ABSTRACT

The process of the present invention is based on the microbial production of enzymes from the growth of the fungus Penicillium funiculosum in a suitable culture medium with a cellulosic substrate. The process of the invention comprises in particular the fermentative treatment of a lignocellulosic substrate by means of a specially adapted fungus, for obtaining an enzymatic preparation capable of hydrolysing cellulose and hemicellulose for the production of ethanol.

### FIELD OF THE INVENTION

The present invention relates to the production of an enzymatic preparation based on a microbiological process, which is capable of hydrolysing the hemicellulosic and cellulosic fractions of the fibres of lignocellulosic residues obtained from forestry and agroindustrial environments. The objective of this hydrolytic process is to generate high concentrations of sugars of the glucose type and significant concentrations of xylose, which can be fermented by microorganisms with the aim of producing ethanol.

### BACKGROUND OF THE INVENTION

Brazil possesses a substantial surplus of agroindustrial and agricultural-forestry residues and is one of the world's major producers of ethanol and cellulose. Consequently, technology that makes it possible to produce ethanol from these surplus lignocellulosic residues, besides helping to solve environmental problems, will add value to these agroindustrial and agricultural-forestry surpluses, generating economic advantages for the country.

For it to be possible to exploit the polysaccharide fractions with a view to production of ethanol, it is necessary for these fractions to be hydrolysed efficiently.

Cracking of these polysaccharides is carried out by means of a pretreatment, which consists of a reaction known as acid hydrolysis, the purpose of which is hydrolysis of the hemicellulosic fraction. The solid resulting from this stage, rich in the cellulosic fraction, still needs to be treated in order to remove the soluble lignin under alkaline conditions to ensure that the cellulosic fibre is accessible to the enzymes.

The conversion of cellulose to ethanol involves two basic steps: hydrolysis of the long chains of the cellulose molecules to sugars (glucose) and fermentation of these sugars to ethanol. In nature, these processes are started by fungi and bacteria, which secrete the enzymes that are able to hydrolyse cellulose (called cellulases), and mainly by yeasts, in the case of the fermentation of sugars to alcohol.

The main difficulty that has to be overcome relates to the microorganism itself, which must be resistant to the operating conditions, mainly with respect to the concentrations of inhibitors produced in the reaction medium.

At present, one of the main bottlenecks in the second-generation biochemical production of ethanol is the production of an enzymatic preparation that is inexpensive and displays good efficiency of hydrolysis of polysaccharides such as cellulose (whose monomer is glucose) and hemicellulose (heteropolymer whose main monomer is xylose).

Production of these enzymes has been the goal of intensive research throughout the world, with the participation of large enterprises (including oil companies) and government organizations, and is fundamental to the development of “clean” technologies.

However, the high cost of these enzymes makes their wide application unviable on a commercial scale and prevents the implementation of industrial plants.

### RELATED TECHNOLOGY

The biotechnological production of ethanol has been investigated since long ago, but has undergone considerable development in recent years. The main obstacle to be overcome relates to productivity, i.e., to achieve a process that is economically viable and has good yield, using raw material that is widely available and of low total value.

Patent document GB 2253633, which corresponds to the Brazilian patent document PI 9200100-9 dated 15 Jan. 1992, describes a process for producing ethanol from biomass, in which the substrate includes a hydrolysate of cellulose, hemicellulose and starch, aiming to produce fermentable six-carbon sugars. Fermentation uses a genetically modified yeast strain (Brettanomyces custersii CBS 5512) that produces the enzyme β-glucosidase, which endows this yeast with the ability to ferment both glucose and cellobiose. However, we are still without a solution to the fermentation of pentoses.

Torget et al. (U.S. Pat. No. 5,705,369) describe a generic process for pre-hydrolysis of lignocellulosic materials, in which various combinations of temperature ranges and reaction time are investigated, with the aim of obtaining better percentage separation of hemicellulose and lignin from cellulose.

Warzwoda et al. (BR 0600409-1) describe a process for production of cellulolytic and hemicellulolytic enzymes from residues (wood from leafy species and cereal straw). Said residues are used as the initial carbon source for obtaining these enzymes, using genetically improved, and notably recombined, strains of Trichoderma reesei. The wild-type strains of this microorganism have the capacity to secrete, in the presence of an inducer substrate (cellulose, for example), the enzymatic complex considered to be the most suitable for the hydrolysis of cellulose. It is therefore a process for production of cellulolytic and/or hemicellulolytic enzymes produced by the specialized strain.

Santa Anna et al. (BR 0505299-8) teach a process for production of ethanol for the purpose of treatment of the solid residue resulting from the acid hydrolysis of sugar cane bagasse. According to this process a hydrolysate of the hemicellulosic fraction of sugar cane bagasse, rich in xylose, was obtained by means of mild hydrolysis with dilute sulphuric acid, and was fermented using a strain of the yeast Pichia stipitis suitably acclimatized to the main substrate of the hydrolysate (xylose). The solid residue resulting from acid hydrolysis (cellulignin) was treated in a special reactor for removal of lignin, by a series of alkaline washings to make the cellulosic fibres suitable for receiving an enzymatic charge.

Santa Anna et al. (BR 0605017-4) describe a process for obtaining ethanol from lignocellulosic materials enzymatically, according to which the hemicellulosic fraction is submitted to mild hydrolysis with sulphuric acid, and the solid material resulting from this hydrolysis is submitted to the process of saccharification (enzymatic hydrolysis) simultaneously with rapid alcoholic fermentation, in conditions permitting a significant increase in conversion to ethanol in much shorter times, using high concentrations of solids (15% to 25%).

Chung and Day (WO 2008/095098) present a process for obtaining sugars from lignocellulosic biomass in which the material is submitted to a hot alkaline pretreatment with a mixture of calcium hydroxide and water at a temperature from 80° C. to 140° C. for about 30 min to 3 hours. After the treatment, the bagasse is pressed; the liquid contains mainly soluble components of lignin, besides lime (which can be recovered) and the fibrous solid material is submitted to hydrolysis by cellulase enzymes. According to the authors, this treatment changes the lignocellulosic structure so that it can be quickly dissolved by cellulase, even using high solids contents (10% to 30%), without affecting the enzymatic activity. Commercial enzymes were used, for example, Spezyme CP (Genecor International Co.) and Novo I88 (Novozyme).

The aim of the process of the present invention is to offer an enzymatic preparation containing enzymes prepared in situ (or dedicated) of low cost, for economically scaling up biochemical technology for application in processes for obtaining ethanol from lignocellulosic materials. The preparation obtained can be applied to the solid produced after hydrolysis of the hemicellulosic fraction as well as fermentation processes that use simultaneous saccharification (SSF), as mentioned above.

### SUMMARY OF THE INVENTION

The process of the present invention is based on the microbial production of enzymes from growth of the fungus in a suitable culture medium with cellulosic substrate.

The invention relates to the production of cellulase enzymes by fermentation using the fungus Penicillium funiculosum, in which synthetic substrates can be used (such as Avicel or carboxymethylcellulose—CMC), or alternatively the actual lignocellulosic and agricultural-forestry residues, in natura or pretreated.

### DETAILED DESCRIPTION OF THE INVENTION

The process of the present invention comprises in particular the fermentative treatment of lignocellulosic and agricultural-forestry substrates by means of a specially adapted fungus, for the purpose of obtaining an enzymatic preparation capable of hydrolysing cellulose and hemicellulose for the production of alcohol.

The invention is based on the production of cellulase enzymes by fermentation using the fungus Penicillium funiculosum ATCC 11797, it being possible to use synthetic substrates, or the actual lignocellulosic and agricultural-forestry residues, in natura or pretreated.

The next stage of the process comprises concentration of the enzymes produced in fermentation, using membrane systems—such as a combined system of microfiltration and ultrafiltration, or by evaporation—for example the rotary evaporator. Finally, an additive is added to this concentrate of enzymes, in order to improve the activity of the enzymes in breaking the cellulose. This additive comprises a biosurfactant of the glycolipid type that promotes an increase in accessibility of the enzymes to cellulose and hemicellulose.

For better understanding of the invention, the process can be described briefly as follows:

The fungus is brought in contact with the cellulosic substrate in a suitable culture medium for a period of four to seven days, so that the enzymes are produced. During this time, the microorganism releases high concentrations of proteins with catalytic properties into the culture medium, i.e. cellulase and hemicellulase enzymes, such as xylanases, endoglucanases, exoglucanases and β-glucosidases, with the aim of breaking the cellulosic and hemicellulosic fractions present in the substrate. After this period, the culture medium containing the enzymes is submitted to a concentration process using membranes or evaporative processes, followed by application of additives.

The enzymatic preparation (extract) was produced with residual materials from conventional production of ethanol, for example sugar cane bagasse and straw, with low production costs, so that it can be produced in situ, with the result that scaling-up of second-generation ethanol production becomes feasible.

Agricultural and forestry material, for example chips and residues from the pulp and paper industry, can also be used as the source of lignocellulose.

The examples given below are for purposes of illustration only, and do not represent any kind of limitation of the invention.

### Example 1

This example aims to demonstrate the potential for use of the process now proposed for obtaining the enzymatic preparation.

Using the process of the invention as already described in detail, it was possible to produce an extract with high concentrations of cellulase enzymes, expressed in enzymatic activities (IU/L) and protein concentrations (mg/L). Table 1 given below presents the results of measurement of enzymatic activity of the extracts at the end of fermentation and after concentration.

TABLE 1

| | FPase | Endoglucanase | β-Glucosidase | Protein |
|---|---|---|---|---|
| EXTRACT | (U/L) | (IU/L) | (IU/L) | (mg/L) |
| Fermented | 125.161 | 2098.922 | 1016.784 | 90.999 |
| Concentrated | 5447.724 | 69276.758 | 40679.012 | 2781.543 |

These concentrated enzymatic extracts were tested in the process of hydrolysis (saccharification) of cellulose and hemicellulose contained in the pretreated sugar cane bagasse, i.e. submitted to mild hydrolysis and washing with heating for removal of lignin, and the results are shown in Table 2.

The measurement of sugars (glucose, xylose and cellobiose) was assessed by the methods of liquid chromatography (HPLC) and spectrophotometric measurement of total reducing sugars (TRS). This table also shows, for comparison, results obtained with a commercial preparation (GENENCOR®), standardized to the same enzyme concentration per gram of cellulose.

TABLE 2

| Enzymatic | Glucose | Xylose | Cellobiose | TRS |
|---|---|---|---|---|
| preparation | (g/L) | (g/L) | (g/L) | (g/L) |
| GENENCOR ® | 36.332 | 0.000 | 2.110 | 45.954 |
| Concentrated | 28.305 | 4.200 | 0.000 | 29.734 |
| extract | | | | |

Table 2 shows the amounts of glucose obtained from the hydrolysis of cellulose. It can also be seen that the concentrated extract of the invention also contains xylanase enzymes that can convert hemicellulose to xylose, which it will also be possible to utilize for conversion to ethanol. This confirms that the enzymatic extract produced can be applied in processes of simultaneous saccharification and fermentation (SSF) and simultaneous saccharification and co-fermentation (SSCF).

### Example 2

The purpose of this example is to demonstrate the applicability of the enzymatic preparation according to the present invention, in the hydrolysis of lignocellulosic residue pretreated by mild hydrolysis and washing with heating to remove the lignin, obtained from the conventional production of ethanol from sugar cane.

The enzymatic preparation was applied in concentrations between 5 and 30 FPU/g, at temperatures between 30° C. and 60° C., for 6 to 48 hours of saccharification.

The concentration of glucose released after the treatment varied from 10 to 40 g/L).

The results achieved enable us to assert that this is a considerable advantage of the extract produced by the process of the invention, by P. funiculosum, in relation to the commercial preparation assessed, as it indicates that the enzyme pool produced can be applied in processes of simultaneous saccharification and co-fermentation, which in terms of equipment is a more integrated and less complex form of processing.

Another advantage of the extract of P. funiculosum is the high β-glucosidase activity, which enables all of the disaccharide cellobiose produced to be converted to glucose (fermentable sugar), whereas with the commercial product there is still a residual amount of cellobiose, a disaccharide that cannot be utilized for obtaining ethanol in the fermentation process.

Another advantage observed is the synergistic action of the enzymes of this preparation, in the hydrolysis of cellulose and hemicellulose, breaking these polysaccharides to sugar monomers, without producing unfermentable residual intermediate sugars, which are not utilized for the production of ethanol.

Furthermore, it was produced from agroindustrial or agricultural-forestry residues that are abundant in Brazil, and applied in situ in the hydrolysis of these same residues, including residues with different compositions, achieving productivity similar to or greater than that resulting from commercial products.

Another great advantage of this invention relates to the high cost of the cellulase enzymes offered in the market, a fact that up to now has prevented the scaling-up of the biochemical technology for conversion of cellulosic materials to ethanol. This invention makes possible the production of these enzymes “in situ” or dedicated, greatly reducing their cost of production.

1. A process for producing an enzymatic preparation, which enzymatic preparation comprises enzymes for the hydrolysis of cellulose from lignocellulosic residues, which process comprises:
bringing a fungus into contact with a cellulosic substrate in a suitable culture medium, optionally for a period of four to seven days, and thereby producing said enzymes;
submitting the culture medium containing said enzymes to a process of concentration, optionally using membranes or an evaporative process; and
optionally adding an additive for improving the activity of said enzymes.

2. A process according to claim 1 which further comprises using the enzymatic extract thus produced in a process for producing fermentable sugars, the process comprising hydrolysing cellulose and/or hemicellulose.

3. A process according to claim 1, wherein the fungus is Penicillium funiculosum.

4. A process according to claim 1, which comprises using the enzymatic extract thus produced in a process comprising saccharification and fermentation, optionally wherein the process comprises simultaneous saccharification and fermentation (SSF), or using the enzymatic extract thus produced in a process comprising saccharification and co-fermentation, optionally wherein the process comprises simultaneous saccharification and co-fermentation (SSCF).

5. A process according to claim 1, which comprises using the enzymatic extract thus produced for the hydrolysis of cellulose from lignocellulosic residues, wherein the lignocellulosic material comprises sugar cane bagasse in natura or pretreated.

6. A process according to claim 5 wherein the lignocellulosic material is pretreated by submitting it to mild hydrolysis and washing with heating to remove the lignin.

7. A process according to claim 5, wherein the lignocellulosic material comprises agricultural-forestry material which comprises chips and/or residues from the papermaking industry.

8. A process according to claim 1, wherein the enzymatic extract produced is used in the hydrolysis of cellulose and/or hemicellulose to produce sugar monomers, without producing unfermentable residual intermediate sugars.

9. A process according to claim 1, wherein the enzymatic preparation is produced in situ, with residual materials from the conventional production of ethanol from sugar cane.

10. A process according to claim 1, wherein the additive is a biosurfactant of the glycolipid type that promotes an increase in accessibility of the enzymes to cellulose and hemicellulose.

11. A process according to claim 1, wherein the enzymatic preparation is produced in situ with residual materials which are in natura and/or pretreated and which comprise residual materials from the conventional production of ethanol from sugar cane, such as bagasse and straw, and/or residual materials from the pulp and paper industry, such as chips.

12. Process for production of an enzymatic preparation for hydrolysis of cellulose from lignocellulosic residues, characterized in that it comprises the stages of:
bringing the fungus in contact with the cellulosic substrate in a suitable culture medium for a period of four to seven days, for the enzymes to be produced;
submitting the culture medium containing the enzymes to a process of concentration using membranes or evaporative processes and application of additives; and
applying the enzymatic extract produced in the process of hydrolysis of the cellulose and hemicellulose for the production of fermentable sugars.

13. A process according to claim 12, wherein the fungus is Penicillium funiculosum.

14. A process according to claim 12, which comprises using the enzymatic extract thus produced in a process comprising saccharification and fermentation, optionally wherein the process comprises simultaneous saccharification and fermentation (SSF), or using the enzymatic extract thus produced in a process comprising saccharification and co-fermentation, optionally wherein the process comprises simultaneous saccharification and co-fermentation (SSCF).

15. A process according to claim 12, which comprises using the enzymatic extract thus produced for the hydrolysis of cellulose from lignocellulosic residues, wherein the lignocellulosic material comprises sugar cane bagasse in natura or pretreated.

16. A process according to claim 6, wherein the lignocellulosic material comprises agricultural-forestry material which comprises chips and/or residues from the papermaking industry.

17. A process according to claim 12, wherein the enzymatic extract produced is used in the hydrolysis of cellulose and/or hemicellulose to produce sugar monomers, without producing unfermentable residual intermediate sugars.

18. A process according to claim 12, wherein the enzymatic preparation is produced in situ, with residual materials from the conventional production of ethanol from sugar cane.

19. A process according to claim 12, wherein the additive is a biosurfactant of the glycolipid type that promotes an increase in accessibility of the enzymes to cellulose and hemicellulose.

20. A process according to claim 12, wherein the enzymatic preparation is produced in situ with residual materials which are in natura and/or pretreated and which comprise residual materials from the conventional production of ethanol from sugar cane, such as bagasse and straw, and/or residual materials from the pulp and paper industry, such as chips.
